(19) United States
(12) Patent Application Publication
(10) Pub. No.: US 2020/0199176 A1
(43) Pub. Date: Jun. 25, 2020

(54) METHOD FOR VIRUS FILTRATION OF VON WILLEBRAND FACTOR

(71) Applicant: CSL BEHRING GMBH, Marburg (DE)

(72) Inventors: Thomas NOWAK, Staufenberg (DE); Holger LIND, Marburg (DE)

(73) Assignee: CSL BEHRING GMBH, Marburg (DE)

(21) Appl. No.: 16/640,568

(22) PCT Filed: Aug. 23, 2018
(86) PCT No.: PCT/EP2018/072710

§ 371 Date: Feb. 20, 2020

(30) Foreign Application Priority Data
Aug. 23, 2017 (EP) 17187436.5

(51) Int. Cl. C07K 1/34; A61K 38/37; A61L 2/00; C07K 14/755; A61L 2/04; A61L 2/08; A61L 2/10; A61L 2/16

(52) U.S. Cl. CPC C07K 1/34; A61K 38/37; A61L 2/0017; C07K 14/755; A61L 2202/21; A61L 2/081; A61L 2/084; A61L 2/10; A61L 2/16; A61L 2/04

(57) ABSTRACT

The present invention relates to a method of filtrating a solution comprising von Willebrand Factor (VWF), the method comprising (a) providing a solution comprising VWF and a basic amino acid; and (b) subjecting the solution of step (a) to a virus filtration through a filter having a pore size of less than or equal to 35 nm.

The present invention relates to a method of filtrating a solution comprising von Willebrand Factor (VWF), the method comprising (a) providing a solution comprising VWF and a basic amino acid; and (b) subjecting the solution of step (a) to a virus filtration through a filter having a pore size of less than or equal to 35 nm.

### BACKGROUND

There are various bleeding disorders caused by deficiencies of blood coagulation factors. The most common disorders are hemophilia A and B, resulting from deficiencies of blood coagulation Factor VIII (FVIII) and IX, respectively. Another known bleeding disorder is von Willebrand's disease (VWD). In plasma FVIII exists mostly as a noncovalent complex with von Willebrand Factor (VWF), and its coagulant function is to accelerate Factor IXa dependent conversion of Factor X to Xa.

VWF, which is missing, functionally defect or only available in reduced quantity in different forms of von Willebrand disease (VWD), is a multimeric adhesive glycoprotein present in the plasma of mammals, which has multiple physiological functions. During primary hemostasis VWF acts as a mediator between specific receptors on the platelet surface and components of the extracellular matrix such as collagen. Moreover, VWF serves as a carrier and stabilizing protein for procoagulant FVIII. VWF is synthesized in endothelial cells and megakaryocytes as a 2813 amino acid precursor molecule. The precursor polypeptide, pre-pro-VWF, consists of an N-terminal 22-residue signal peptide, followed by a 741-residue pro-peptide and the 2050-residue polypeptide found in mature plasma VWF (Fischer et al., FEBS Lett. 351: 345-348, 1994). After cleavage of the signal peptide in the endoplasmatic reticulum a C-terminal disulfide bridge is formed between two monomers of VWF. During further transport through the secretory pathway 12 N-linked and 10 O-linked carbohydrate side chains are added. More important, VWF dimers are multimerized via N-terminal disulfide bridges and the propeptide of 741 amino acids length is cleaved off by the enzyme PACE/furin in the late Golgi apparatus.

Once secreted into plasma the protease ADAMTS13 can cleave high-molecular weight VWF multimers within the A1 domain of VWF. Plasma VWF therefore consists of a whole range of multimers ranging from single dimers of 500 kDa to multimers consisting of up to more than 20 dimers of a molecular weight of over 10,000 kDa. The VWF high molecular weight multimers (HMWM) hereby having the strongest hemostatic activity, which can be measured in ristocetin cofactor activity (VWF:RCo). The higher the ratio of VWF:RCo/VWF antigen, the higher the relative amount of high molecular weight multimers.

Various methods of purifying VWF or FVIII/VWF complex have been described, e.g. in U.S. Pat. No. 5,854,403, EP0411810A1, EP0639203 or Ristol P. et al., Sangre (1996) 41:125-130.

Purification of VWF requires one or more steps for removing potentially present pathogens, e.g. viruses. One method which is very effective in eliminating viruses is filtration through filters having a pore size capable of holding back viral particles (virus filtration). The efficacy of this method depends on the pore size of the filter that is used.

There are nanofilters of different pore sizes, normally between 15 and 75 nanometers (nm), and a smaller pore size results in a greater effectiveness in retaining pathogens. Nanofilters having a pore size below 35 nm and preferably between 15 and 20 nm are able to remove even very small viruses such as erythrovirus B19 or hepatitis A virus. Filtration through a nanofilter having a small pore size, however, is problematic if proteins of high molecular weight should also pass the filter (WO2005/040214A1). Generally, VWF or the FVIII/VWF complex does not appear suitable for efficient filtration through nanofilters having a pore size of less than 35 nm, especially if the VWF solution comprises the multimer forms of VWF of higher molecular weight (EP1632501).

EP1348445A1 describes a process for separating viruses from a solution comprising fibrinogen by nanofiltration, wherein a chaotropic agent is added to the fibrinogen solution prior to the nanofiltration. EP1348445A1, however, does not mention VWF.

EP2078730A1 describes a process wherein a solution containing VWF or the FVIII/VWF complex can be filtered through a nanofilter of nominal pore size less than 35 nm and even 20 nm if calcium ions are present (see paragraph [0033] of EP2078730A1).

WO2015/188224A1 describes a process for manufacturing recombinant VWF which comprises separating the multimers of VWF into a permeate fraction enriched in low molecular weight multimers of VWF and a retentate fraction enriched in HMWM of VWF. The pore size of the filters, however, is relatively large (0.05 μm to 1 μm).

The prior art methods are still unsatisfactory with regard to the yield of the VWF in the filtrate.

Thus, there is an ongoing need for improved methods for virus filtration of VWF, in particular for methods giving a good yield of VWF.

### SUMMARY OF THE INVENTION

The inventors of the present application found that a surprisingly high yield in VWF antigen and VWF activity is obtained in the filtrate upon virus filtration of a VWF solution if the virus filtration is carried out in the presence of at least 150 mM arginine. It was further found that similar results are obtained with lysine and histidine. Therefore, the present invention inter alia relates to the aspects and embodiments defined in items [1] to [64] hereinafter.
[1] A method of filtrating a solution comprising von Willebrand Factor (VWF), the method comprising the following steps:
   (a) providing a solution comprising VWF and at least one basic amino acid at a concentration of at least 150 mM;
   (b) subjecting the solution of step (a) to a virus filtration through a filter having a pore size of less than or equal to 35 nm.
[2] The method of item [1], wherein said VWF in the solution of step (a) comprises high molecular weight multimers (HMWM) of VWF.
[3] The method according to item [1] or [2], wherein the pressure during the virus filtration in step (b) is below 0.5 bar.
[4] The method of item [3], wherein the pressure during the virus filtration in step (b) is from 0.1 to 0.4 bar.
[5] The method according to any one of the preceding items, wherein the pH of the solution provided in step (a) is between 5.0 and 9.0.
[6] The method according to any one of the preceding items, wherein the pH of the solution provided in step (a) is between 6.0 and 8.0.
[7] The method according to any one of the preceding items, wherein the pH of the solution provided in step (a) is between 6.5 and 7.5.
[8] The method according to any one of the preceding items, wherein the virus filtration in step (b) is conducted at a temperature between 15 and 30° C.
[9] The method according to any one of the preceding items, wherein the virus filtration in step (b) is conducted at a temperature between 18 and 28° C.
[10] The method according to any one of the preceding items, wherein the concentration of said at least one basic amino acid in the solution provided in step (a) is at least 300 mM.
[11] The method according to any one of the preceding items, wherein the concentration of said at least one basic amino acid in the solution provided in step (a) is at least 350 mM.
[12] The method according to any one of the preceding items, wherein the concentration of said at least one basic amino acid in the solution provided in step (a) is at least 400 mM.
[13] The method according to any one of the preceding items, wherein the concentration of said at least one basic amino acid in the solution provided in step (a) is at least 450 mM.
[14] The method according to any one of the preceding items, wherein the concentration of said at least one basic amino acid in the solution provided in step (a) is at least 500 mM.
[15] The method according to any one of the preceding items, wherein the concentration said at least one basic amino acid in the solution provided in step (a) is less than 1,000 mM.
[16] The method according to any one of the preceding items, wherein the concentration of said at least one basic amino acid in the solution provided in step (a) is less than 900 mM.
[17] The method according to any one of the preceding items, wherein the concentration of said at least one basic amino acid in the solution provided in step (a) is less than 800 mM.
[18] The method according to any one of the preceding items, wherein the concentration of said at least one basic amino acid in the solution provided in step (a) is less than 750 mM.
[19] The method according to any one of the preceding items, wherein the solution provided in step (a) further comprises calcium ions at a concentration of at least 50 mM.
[20] The method according to any one of the preceding items, wherein the solution provided in step (a) further comprises calcium ions at a concentration of at least 100 mM.
[21] The method according to any one of the preceding items, wherein the solution provided in step (a) further comprises calcium ions at a concentration of at least 200 mM.
[22] The method according to any one of the preceding items, wherein the solution provided in step (a) further comprises calcium ions at a concentration of at least 300 mM.
[23] The method according to any one of the preceding items, wherein the solution provided in step (a) further comprises calcium ions at a concentration of at least 350 mM.
[24] The method according to any one of the preceding items, wherein the filter has a median pore size of less than or equal to 35 nm.
[25] The method according to any one of the preceding items, wherein the filter has a median pore size of less than or equal to 25 nm.
[26] The method according to any one of the preceding items, wherein the filter has a median pore size of less than or equal to 20 nm.
[27] The method according to any one of the preceding items, wherein the filter has a median pore size of between 13 nm and 35 nm.
[28] The method according to any one of the preceding items, wherein the filter has a median pore size of between 13 nm and 25 nm.
[29] The method according to any one of the preceding items, wherein the filter has a median pore size of between 18 nm and 22 nm.
[30] The method according to any one of the preceding items, wherein the filter has a median pore size of between 13 nm and 17 nm.
[31] The method according to any one of the preceding items, wherein the VWF is plasma-derived VWF.
[32] The method according to any one of items [1] to [30], wherein the VWF is recombinantly obtained VWF.
[33] The method of item [32], wherein the VWF comprises a half-life extending moiety.
[34] The method of item [33], wherein said half-life extending moiety is a heterologous amino acid sequence fused to a VWF amino acid sequence.
[35] The method of item [33], wherein said heterologous amino acid sequence comprises or consists of a polypeptide selected from the group consisting of immunoglobulin constant regions and portions thereof, e.g. the Fc fragment, transferrin and fragments thereof, the C-terminal peptide of human chorionic gonadotropin, solvated random chains with large hydrodynamic volume known as XTEN, homo-amino acid repeats (HAP), proline-alanine-serine repeats (PAS), albumin, afamin, alpha-fetoprotein, Vitamin D binding protein, polypeptides capable of binding under physiological conditions to albumin or immunoglobulin constant regions, and combinations thereof.
[36] The method of item [33], wherein said half-life extending moiety is conjugated to the polypeptide.
[37] The method of item [36], wherein said half-life-extending moiety is selected from the group consisting of hydroxyethyl starch (HES), polyethylene glycol (PEG), polysialic acids (PSAs), elastin-like polypeptides, heparosan polymers, hyaluronic acid and albumin binding ligands, e.g. fatty acid chains, and combinations thereof.
[38] The method according to any one of the preceding items, wherein the ratio RCo VWF/Ag VWF in the filtrate obtained in step (b) is at least 0.75.
[39] The method according to any one of the preceding items, wherein the ratio RCo VWF/Ag VWF in the filtrate obtained in step (b) is at least 0.8.
[40] The method according to any one of the preceding items, wherein the ratio RCo VWF/Ag VWF in the filtrate obtained in step (b) is at least 0.9.
[41] The method according to any one of the preceding items, wherein the ratio RCo VWF/Ag VWF in the filtrate obtained in step (b) is at least 1.0.
[42] The method according to any one of the preceding items, wherein the ratio RCo VWF/Ag VWF in the filtrate obtained in step (b) is at least 1.1.
[43] The method according to any one of the preceding items, wherein the ratio RCo VWF/Ag VWF in the filtrate obtained in step (b) is at least 1.2.
[44] The method according to any one of the preceding items, wherein the ratio RCo VWF/Ag VWF in the filtrate obtained in step (b) is at least 75% of the ratio RCo VWF/Ag VWF in the solution provided in step (a).
[45] The method according to any one of the preceding items, wherein the VWF:Ag yield following filtration is at least 50%.
[46] The method according to any one of the preceding items, wherein the VWF:Ag yield following filtration is at least 60%.
[47] The method according to any one of the preceding items, wherein the VWF:Ag yield following filtration is at least 70%.
[48] The method according to any one of the preceding items, wherein the VWF:Ag yield following filtration is at least 75%.
[49] The method according to any one of the preceding items, wherein the RCo VWF yield following filtration is at least 40%.
[50] The method according to any one of the preceding items, wherein the RCo VWF yield following filtration is at least 45%.
[51] The method according to any one of the preceding items, wherein the RCo VWF yield following filtration is at least 50%.
[52] The method according to any one of the preceding items, wherein the RCo VWF yield following filtration is at least 55%.
[53] The method according to any one of the preceding items, wherein the solution provided in step (a) as well as the filtrate obtained in step (b) comprises when analysed by multimer electrophoresis low multimers (1-5 bands), intermediate multimers (6-10 bands) and large multimers (HMWM, high molecular weight multimers, higher than 11 bands) of VWF, provided that the relative amount of large multimers in the filtrate obtained in step (b) is at least 70% when compared to the total VWF content in the solution provided in step (a) and in the filtrate obtained in step (b), respectively.
[54] The method of item [53], wherein the relative amount of large multimers in the filtrate obtained in step (b) is at least 75%.
[55] The method of item [53], wherein the relative amount of large multimers in the filtrate obtained in step (b) is at least 80%.
[56] The method of item [53], wherein the relative amount of large multimers in the filtrate obtained in step (b) is at least 85%.
[57] The method according to any one of the preceding items, wherein said at least one amino acid is selected from the group consisting of arginine, lysine, histidine, ornithine and combinations thereof.
[58] The method according to any one of the preceding items, wherein said at least one amino acid is arginine.
[59] The method according to any one of items [1] to [57], wherein said at least one amino acid is lysine.
[60] The method according to any one of items [1] to [57], wherein said at least one amino acid is histidine.
[61] The method according to any one of the preceding items, wherein the solution provided in step (a) comprises Factor VIII (FVIII) in addition to VWF, wherein the solution provided in step (a) may preferably comprise a complex of VWF and FVIII.
[62] A filtrated solution of VWF obtainable by a method according to any one of the preceding items.
[63] A composition comprising VWF obtainable by a method according to any one of preceding items.
[64] A process of purifying VWF, comprising the method of any one of items [1] to [61].

### DETAILED DESCRIPTION

In a first aspect, the present invention relates to a method of filtrating a solution comprising VWF. The method comprises (a) providing a solution comprising VWF and at least 150 mM of a basic amino acid; and (b) subjecting the solution of step (a) to a virus filtration through a filter having a pore size of less than or equal to 35 nm.

Von Willebrand Factor

The term “von Willebrand Factor” or “VWF”, as used herein, refers to any polypeptide having the biological activity of wild type VWF or at least a partial biological activity of VWF.

By “biological activity” a measurable function of VWF is meant which VWF performs also in vivo when administered to a human being. As used herein, the terms “function” and “functional” and the like refer to a biological, enzymatic, or therapeutic function of VWF. The biological activity of VWF can for example be determined by the artisan using methods to determine the ristocetin co-factor activity (VWF:RCoF) (Federici A B et al. 2004. Haematologica 89:77-85), the binding of VWF to GP lb of the platelet glycoprotein complex lb-V-IX (Sucker et al. 2006. Clin Appl Thromb Hemost. 12:305-310), a collagen binding assay (Kailas & Talpsep. 2001. Annals of Hematology 80:466-471) or a FVIII binding assay. FVIII binding may be determined for example by Biacore analysis.

The term “von Willebrand Factor” (VWF) includes naturally occurring (native) VWF, but also variants thereof having at least part of the biological activity of naturally occurring VWF, e.g. sequence variants where one or more residues have been inserted, deleted or substituted. The gene encoding wild type VWF is transcribed into a 9 kb mRNA which is translated into a pre-propolypeptide of 2813 amino acids with an estimated molecular weight of 310,000 Da. The pre-propolypeptide consists of 2813 amino acids and contains a 22 amino acids signal peptide, a 741 amino acid pro-polypeptide and the mature subunit. Cleavage of the 741 amino acids pro-polypeptide from the N-terminus results in mature VWF consisting of 2050 amino acids. The cDNA sequence of wild type pre-pro-VWF is shown in SEQ ID NO:1. The amino acid sequence of wild type pre-pro-VWF is shown in SEQ ID NO:2. The term “VWF” as used herein refers to the mature form of VWF unless indicated otherwise.

Preferably, wild type VWF comprises the amino acid sequence of wild type VWF as shown in SEQ ID NO:2. Also encompassed are additions, insertions, N-terminal, C-terminal or internal deletions of VWF as long as at least a partial biological activity of VWF is retained.

In a preferred embodiment the VWF is a plasma-derived VWF, more preferred a human plasma-derived VWF.

In certain embodiments of the method of the invention, the VWF is recombinantly produced wild-type VWF as for example described in WO2010/048275A2, or a variant thereof, for example, in which one or more amino acid deletions, additions, and/or substitutions have been introduced to increase or decrease at least one biological activity of the protein.

Accordingly, certain embodiments may employ any one or more of these VWF-related sequences, including combinations and variants thereof. Also included are VWF-related sequences from other organisms, such as other mammals described herein and known in the art.

In certain embodiments the term “VWF” includes fusion proteins of VWF, preferably fusion proteins of a VWF protein and a heterologous fusion partner. Also included are fusion proteins or modified proteins that comprise a heterologous fusion partner or heterologous sequence and at least one minimal fragment or portion of a VWF protein.

As used herein, a “fusion protein” includes a VWF protein or fragment thereof linked to either another (e.g., different) VWF protein (e.g., to create multiple fragments), to a non-VWF protein, or to both. A “non-VWF protein” refers to a “heterologous polypeptide” having an amino acid sequence corresponding to a protein which is different from a wild-type VWF protein, and which can be derived from the same or a different organism. The VWF portion of the fusion protein can correspond to all or a fragment of a biologically active VWF protein amino acid sequence. In certain embodiments, a VWF fusion protein includes at least one (or two, three, etc.) biologically active portion(s) of a VWF protein.

More generally, fusion to heterologous sequences, such as albumin or immunoglobulins or fragments derived from immunoglobulins without an antigen binding domain, such as the Fc fragment, may be utilized to remove unwanted characteristics or to improve the desired characteristics (e.g., pharmacokinetic properties) of a VWF. For example, fusion to a heterologous sequence may increase chemical stability, decrease immunogenicity, improve in vivo targeting, and/or increase half-life in circulation of a VWF protein.

Suitable heterologous sequences that may be fused with a VWF sequence include, but are not limited to, immunoglobulin constant regions and portions thereof, e.g. the Fc fragment, transferrin and fragments thereof, the C-terminal peptide of human chorionic gonadotropin, solvated random chains with large hydrodynamic volume known as XTEN, homo-amino acid repeats (HAP), proline-alanine-serine repeats (PAS), albumin, afamin, alpha-fetoprotein, Vitamin D binding protein, polypeptides capable of binding under physiological conditions to albumin or immunoglobulin constant regions, and combinations thereof.

“Albumin”, as used herein, includes polypeptides of the albumin family of proteins such as human serum albumin and bovine serum albumin, including variants and derivatives thereof, such as genetically engineered or chemically modified albumin variants and fragments of albumin proteins. The albumin portion of a fusion protein may be derived from any vertebrate, especially any mammal, for example human, cow, sheep, or pig. Non-mammalian albumins include, but are not limited to, hen and salmon. The albumin portion of the albumin-linked polypeptide may be from a different animal than the VWF protein portion of the fusion protein. Preferably the albumin is human serum albumin.

The albumin family of proteins, included within the term “albumin” used herein, comprise evolutionarily related serum transport proteins, for example, albumin, alpha-fetoprotein (AFP; Beattie & Dugaiczyk, Gene. 20:415-422, 1982), afamin (AFM; Lichenstein et al., J. Biol. Chem. 269: 18149-18154, 1994), and vitamin D binding protein (DBP; Cooke & David, J. Clin. Invest. 76:2420-2424, 1985). Alpha-fetoprotein has been claimed to enhance the half-life of an attached therapeutic polypeptide (see WO2005/024044A2). Their genes represent a multigene cluster with structural and functional similarities mapping to the same chromosomal region in humans, mice and rat. Some embodiments of the invention, therefore, may use such albumin family members, or fragments and variants thereof as defined herein, as part of a fusion protein. Albumin family members of the therapeutic fusion proteins of the invention may also include naturally-occurring polymorphic variants of AFP, AFM and DBP.

VWF protein, or a fragment or variant thereof, may be fused to a human serum albumin polypeptide, or a fragment or variant thereof (see, e.g. WO2009/156137A1). Human serum albumin (HSA, or HA) is a protein of 585 amino acids in its mature form and is responsible for a significant proportion of the osmotic pressure of serum and also functions as a carrier of endogenous and exogenous ligands. Among other benefits, fusion to HSA or a fragment or variant thereof can increase the shelf-life, serum half-life, and/or therapeutic activity of the VWF proteins described herein.

Preferably a fusion protein comprises albumin as the C-terminal portion, and a VWF protein as the N-terminal portion. In other embodiments, the fusion protein has VWF proteins fused to both the N-terminus and the C-terminus of albumin.

In a preferred embodiment the VWF in accordance with the invention is a VWF-albumin fusion protein as disclosed in WO2009/156137A1.

A peptide linker sequence may be employed to separate the components of a fusion protein. For instance, peptide linkers can separate the components by a distance sufficient to ensure that each polypeptide folds into its secondary and tertiary structures. Such a peptide linker sequence may be incorporated into the fusion protein using standard techniques described herein and well-known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. Nos. 4,935,233 and 4,751,180.

One or more of the non-peptide or peptide linkers are optional. For instance, linker sequences may not be required in a fusion protein where the first and second polypeptides have non-essential N-terminal and/or C-terminal amino acid regions that can be used to separate the functional domains and prevent steric interference.

Certain embodiments of the present invention also contemplate the use of modified VWF proteins, including modifications that improved the desired characteristics of the protein, as described herein. Modifications of VWF proteins include chemical and/or enzymatic derivatizations at one or more constituent amino acid, including side chain modifications, backbone modifications, and N- and C-terminal modifications including acetylation, hydroxylation, methylation, amidation, and the attachment of carbohydrate or lipid moieties, cofactors, and the like. Exemplary modifications also include PEGylation of a VWF protein (see, e.g., Veronese and Harris, Advanced Drug Delivery Reviews 54: 453-456, 2002, herein incorporated by reference). VWF variants which are chemically conjugated to biologically acceptable polymers are described for example in WO2006/071801A2.

In certain embodiments, a half-life extending moiety is conjugated to the VWF portion of the polypeptide. Suitable half-life extending moieties include, but are not limited to, hydroxyethyl starch (HES), polyethylene glycol (PEG), polysialic acids (PSAs), elastin-like polypeptides, heparosan polymers, hyaluronic acid and albumin binding ligands, e.g. fatty acid chains, and combinations thereof.

The invention may also be used with “variants” of VWF proteins. The term protein “variant” includes proteins that are distinguished from SEQ ID NO:2 by the addition, deletion, and/or substitution of at least one amino acid residue, and which typically retain one or more activities of the reference protein. It is within the skill of those in the art to identify amino acids suitable for substitution and to design variants with substantially unaltered, improved, or decreased activity, relative to a reference sequence.

A protein variant may be distinguished from a reference sequence by one or more substitutions, which may be conservative or non-conservative, as described herein and known in the art. In certain embodiments, the protein variant comprises conservative substitutions and, in this regard, it is well understood in the art that some amino acids may be changed to others with broadly similar properties without changing the nature of the activity of the protein.

As noted above, biologically active variant proteins may contain conservative amino acid substitutions at various locations along their sequence, as compared to a reference residue.

A “conservative amino acid substitution” includes one in which the amino acid residue is replaced with an amino acid residue having a similar side chain. Families of amino acid residues having similar side chains have been defined in the art, which can be generally sub-classified as follows:

Acidic: The residue has a negative charge due to loss of H ion at physiological pH and the residue is attracted by aqueous solution so as to seek the surface positions in the conformation of a peptide in which it is contained when the peptide is in aqueous medium at physiological pH. Amino acids having an acidic side chain include glutamic acid and aspartic acid.

Basic: The residue has a positive charge due to association with H ion at physiological pH or within one or two pH units thereof (e.g., histidine) and the residue is attracted by aqueous solution so as to seek the surface positions in the conformation of a peptide in which it is contained when the peptide is in aqueous medium at physiological pH. Amino acids having a basic side chain include arginine, lysine and histidine.

Charged: The residues are charged at physiological pH and, therefore, include amino acids having acidic or basic side chains (i.e., glutamic acid, aspartic acid, arginine, lysine and histidine).

Hydrophobic: The residues are not charged at physiological pH and the residue is repelled by aqueous solution so as to seek the inner positions in the conformation of a peptide in which it is contained when the peptide is in aqueous medium. Amino acids having a hydrophobic side chain include tyrosine, valine, isoleucine, leucine, methionine, phenylalanine and tryptophan.

Neutral/polar: The residues are not charged at physiological pH, but the residue is not sufficiently repelled by aqueous solutions so that it would seek inner positions in the conformation of a peptide in which it is contained when the peptide is in aqueous medium. Amino acids having a neutral/polar side chain include asparagine, glutamine, cysteine, histidine, serine and threonine.

This description also characterizes certain amino acids as “small” since their side chains are not sufficiently large, even if polar groups are lacking, to confer hydrophobicity. With the exception of proline, “small” amino acids are those with four carbons or less when at least one polar group is on the side chain and three carbons or less when not. Amino acids having a small side chain include glycine, serine, alanine and threonine. The gene-encoded secondary amino acid proline is a special case due to its known effects on the secondary conformation of peptide chains. The structure of proline differs from all the other naturally-occurring amino acids in that its side chain is bonded to the nitrogen of the α-amino group, as well as the α-carbon. For the purposes of the present invention, proline is classified as a “small” amino acid.

The degree of attraction or repulsion required for classification as polar or nonpolar is arbitrary and, therefore, amino acids specifically contemplated by the invention have been classified as one or the other. Most amino acids not specifically named can be classified on the basis of known behavior.

Amino acid residues can be further sub-classified as cyclic or non-cyclic, and aromatic or non-aromatic, self-explanatory classifications with respect to the side-chain substituent groups of the residues, and as small or large. The residue is considered small if it contains a total of four carbon atoms or less, inclusive of the carboxyl carbon, provided an additional polar substituent is present; three or less if not. Small residues are, of course, always non-aromatic. Dependent on their structural properties, amino acid residues may fall in two or more classes. For the naturally-occurring protein amino acids, sub-classification according to this scheme is presented in Table 1 below.

TABLE 1
Amino acid sub-classification

| Sub-dasses | Amino acids |
|---|---|
| Acidic | Aspartic acid, Glutamic acid |
| Basic Charged | Noncyclic: Arginine, Lysine; Cyclic: Histidine |
| Small | |
| Polar/neutral | Aspartic acid, Glutamic acid, Arginine, Lysine, |
| | Histidine |
| Polar/large | Glycine, Serine, Alanine, Threonine, Proline |
| Hydrophobic | Asparagine, Histidine, Glutamine, Cysteine, |
| | Serine, Threonine Asparagine, Glutamine |
| Aromatic | Tyrosine, Valine, |

Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide. Whether an amino acid change results in a biologically active protein can readily be determined by assaying its chromogenic and/or coagulation activity, as described herein.

VWF Solution

The solution referred to in step (a) of the method of the invention comprises VWF and at least 150 mM of a basic amino acid or of a combination of basic amino acids.

The VWF in the solution to be filtrated preferably comprises high molecular weight multimers (HMWM) of VWF.

The terms “high molecular weight VWF multimers” or “HMW VWF multimers” or “HMWM of VWF” are used synonymously and are meant to correspond to bands 11 and higher in a densidometric VWF analysis according to Ott et al. (Am J Clin Pathol 2010; 133:322-330), wherein “higher” means band 11 and all larger VWF multimers.

The terms “low molecular weight VWF multimers” or “low multimers” or “LMWM of VWF” are used synonymously and are meant to correspond to bands 1 to 5 in a densidometric VWF analysis according to Ott et al. (Am J Clin Pathol 2010; 133:322-330).

The terms “intermediate molecular weight VWF multimers” or “intermediate multimers” or “IMWM of VWF” are used synonymously and are meant to correspond to bands 6 to 10 in a densidometric VWF analysis according to Ott et al. (Am J Clin Pathol 2010; 133:322-330).

The VWF concentration (Ag VWF) in the solution to be filtrated may range from 0.1 to 30 IU/ml, preferably it ranges from 1 to 25, or from 3 to 20, or from 5 to 15 IU/ml.

The VWF concentration (RCo VWF) in the solution to be filtrated may range from 0.1 to 30 IU/ml, preferably it ranges from 1 to 25, or from 3 to 20, or from 5 to 15 IU/ml.

The ratio RCo VWF/Ag VWF in the solution to be filtrated is preferably at least 0.75, or at least 0.8, or at least 0.9, or at least 1.0, or at least 1.1, or at least 1.2. Typically, the ratio RCo VWF/Ag VWF in the solution to be filtrated ranges from 0.5 to 2, preferably from 0.75 to 1.8, or from 0.8 to 1.6.

The VWF solution to be filtrated may also comprise Factor VIII (FVIII). The FVIII concentration in the solution to be filtrated may range from about 0.1 IU/ml to about 20 IU/ml, or from about 1 IU/ml to about 10 IU/ml. The FVIII may be present as a complex with VWF.

Basic Amino Acid

The term “basic amino acid” as used herein refers to an amino acid having an isoelectric point greater than 7.

The VWF solution to be filtrated may comprise one basic amino acid at a concentration of at least 150 mM, or a combination of basic amino acids, wherein the total concentration of all basic amino acids in the VWF solution is at least 150 mM. For example, the VWF solution may comprise 150 mM arginine, or it may comprise 75 mM arginine and 75 mM lysine so that the overall concentration of basic amino acids is 150 mM. Both embodiments are within the scope of this invention.

Preferably, the basic amino acid is selected from the group consisting of arginine, lysine, histidine, ornithine and combinations thereof. More preferably, the basic amino acid is selected from the group consisting of arginine, lysine, histidine and combinations thereof.

In a preferred embodiment, the basic amino acid is arginine. More preferably, arginine is the sole basic amino acid in the VWF solution to be filtrated.

In another embodiment, the basic amino acid is lysine. Preferably, lysine is the sole basic amino acid in the VWF solution to be filtrated.

In another embodiment, the basic amino acid is histidine. Preferably, lysine is the sole basic amino acid in the VWF solution to be filtrated.

In yet another embodiment, the basic amino acid is a combination of arginine and lysine.

In yet another embodiment, the basic amino acid is a combination of arginine and histidine.

In yet another embodiment, the basic amino acid is a combination of histidine and lysine.

In yet another embodiment, the basic amino acid is a combination of arginine, lysine and histidine.

The concentration of the at least one basic amino acid in the solution to be filtrated is preferably at least 200 mM, or at least 250 mM, or at least 300 mM, or at least 350 mM, or at least 400 mM, or at least 450 mM, or at least 500 mM. It is further preferred that the concentration of the at least one basic amino acid in the solution to be filtrated is less than 1,000 mM, or less than 950 mM, or less than 900 mM, or less than 850 mM, or less than 800 mM, or less than 750 mM, or less than 700 mM. In other embodiments the concentration of the at least one basic amino acid in the solution to be filtrated ranges from 150 mM to 1,000 mM, or from 200 mM to 950 mM, or from 250 mM to 900 mM, or from 300 mM to 850 mM, or from 350 mM to 800 mM. Most preferably, the concentration of the at least one basic amino acid in the solution to be filtrated ranges from 400 mM to 800 mM, e.g. from 450 mM to 750 mM, or from 500 mM to 700 mM. Particularly suitable concentrations include about 400 mM, about 450 mM, about 500 mM, about 550 mM, about 600 mM, about 650 mM, about 700 mM, and about 750 mM.

The solution to be filtrated may comprise further compounds, in addition to the VWF and the at least one basic amino acid. In a preferred embodiment, the solution to be filtrated further comprises calcium ions (Ca²⁺). The concentration of the calcium ions in the solution to be filtrated is preferably at least 50 mM, or at least 100 mM, or at least 150 mM, or at least 200 mM, or at least 250 mM, or at least 300 mM, or at least 350 mM. Preferably, the concentration of the calcium ions in the solution to be filtrated ranges from 50 mM to 800 mM, or from 100 mM to 750 mM, or from 150 mM to 700 mM, or from 200 mM to 650 mM, or from 250 mM to 600 mM, or from 300 mM to 550 mM, or from 350 mM to 500 mM.

The VWF solution to be filtrated may comprises additional compounds, including, but not limited to, alkali metal salts, amino acids, and buffer substances. Preferred additional compounds include sodium chloride (NaCl), glycin, histidine, sodium citrate, MES and HEPES.

Most preferably, the solution to be filtrated comprises VWF, 400 mM to 800 mM arginine, and 300 mM to 500 mM CaCl₂).

The solution to be filtrated typically has a pH in the range from 6.0 to 8.0. Preferably, the pH of the solution is from 6.1 to 7.8, or from 6.2 to 7.6, or from 6.3 to 7.4, or from 6.4 to 7.2. More preferably, the pH of the solution is from 6.5 to 7.1. Most preferably, the pH is from 6.6 to 7.0, or from 6.7 to 6.9, e.g. about 6.8. The pH can be adjusted and maintained by the use of suitable buffer substances, e.g. MES or HEPES.

The protein concentration in the solution to be filtrated is typically in the range from about 0.01 mg/ml to about 1 mg/ml, preferably from about 0.05 mg/ml to about 0.8 mg/ml, more preferably from about 0.1 mg/ml to about 0.5 mg/ml.

The Filter

The filter used in the method of the present invention has a nominal pore size of 35 nm or less. Preferably, the nominal pore size of the filter is 25 nm or less. More preferably, the nominal pore size of the filter is 22 nm or less. Most preferably the nominal pore size of the filter is 20 nm or less, e.g. 15 nm, 16 nm, 17 nm, 18 nm or 19 nm. The nominal pore size of the filter used in the method of the present invention is preferably in the range from 15 nm to 35 nm, or from 16 nm to 30 nm, or from 17 nm to 25 nm, or from 18 nm to 22 nm.

The filter used in the method of the present invention has a median pore size of 35 nm or less. Preferably, the median pore size of the filter is 25 nm or less. More preferably, the median pore size of the filter is 22 nm or less. Most preferably the median pore size of the filter is 20 nm or less, e.g. 15 nm, 16 nm, 17 nm, 18 nm or 19 nm. The median pore size of the filter used in the method of the present invention is preferably in the range from 15 nm to 35 nm, or from 16 nm to 30 nm, or from 17 nm to 25 nm, or from 18 nm to 22 nm.

The membrane of the filter can be made of different materials. Preferably, the membrane comprises or substantially consists of polyethersulfone (such as, e.g., Sartorius Virosart® CPV), hydrophilic, optionally modified, polyvinylidenedifluoride (such as, e.g., Pall Pegasus™ SV4), or cellulose, e.g. cuprammonium regenerated cellulose (such as, e.g., AsahiKasei Planova 20N).

Suitable filters include, but are not limited to, Sartorius Virosart® CPV, Pall Pegasus™ SV4 and AsahiKasei Planova 20N. Several suitable filters are summarized in Table 2 below.

TABLE 2
Parvovirus-Grade Filters

| Company | Virus | | |
|---|---|---|---|
| (alphabetical) | filter | Membrane chemistry | Area [m²] |
| Asahi | Planova | cuprammon regeneraterd | 0.001 |
| | 15N/20N | cellulose, hydrophilic, | |
| | | hollow fiber | |
| | Planova | modified PVDF | 0.001 |
| | BioEx | (Polyvinylidene | |
| | | Fluoride), hollow | |
| | | fiber | |
| Millipore | Virosolve | hydrophilic PES | 0.00031 |
| | Pro | (Polyethersulfone), | |
| | | double layer | |
| | Viresolve | hydrophilic PVDF | 0.00035 |
| | NFP | (Polyvinylidene | |
| | | Fluoride), triple | |
| | | layer | |
| Pall | DV20 | hydrophilic modified | 0.00096 |
| | | acrylate PVDF | |
| | | (Polyvinylidene | |
| | | Fluoride), double | |
| | | layer | |
| | Pegasus | hydrophilic modified | 0.00096 |
| | SV4 | acrylate PVDF | |
| | | (Polyvinylidene | |
| | | Fluoride), double | |
| | | layer | |
| | Pegasus | hydrophilic | 0.00028 |
| | Prime | modified PES | |
| | | (Polyethersulfone) | |
| Sartorius | Virosart | hydrophilic PES | 0.0005 |
| | CPV | (Polyethersulfone), | |
| | | double layer | |
| | Virosart | hydrophilic | 0.0005 |
| | HF | modified PES | |
| | | (Polyethersulfone), | |
| | | single layer | |
| | Virosart | hydrophilic | 0.0005 |
| | HC | modified PES | |
| | | (Polyethersulfone), | |
| | | double layer | |

The effective surface of the filter membrane may range from about 0.001 m² to about 10 m², or from about 0.01 m² to about 4 m², or from about 0.1 m² to about 1 m².

The Filtration Process

Typically, the filtration according to the method of the present invention is carried out as dead-end filtration. The volume of the solution to be filtration may range from 10 mL to 100 L, or from 100 ml to 10 L or from 0.5 L to 5 L.

The temperature of the solution to be filtrated at the beginning of the filtration and during the filtration process may range from about 10° C. to about 30° C. Preferably, the temperature of the solution to be filtrated at the beginning of the filtration and during the filtration process is from 15° C. to 29° C., or from 18° C. to 28° C., e.g. about 19° C., about 20° C., about 21° C., about 22° C., about 23° C., about 24° C., about 25° C., about 26° C., or about 27° C.

The filtration is typically carried out at a pressure of less than 1 bar. Preferably, the filtration is carried out at a pressure of less than 0.75 bar. More preferably, the filtration is carried out at a pressure of less than 0.5 bar. Most preferably, the filtration is carried out at a pressure of from 0.1 bar to 0.45 bar, or from 0.2 bar to 0.4 bar, e.g. about 0.3 bar.

The filtration flow may range from about 1 L/hour/m² to about 30 L/hour/m². Preferably, the filtration is from about 5 L/hour/m² to about 25 L/hour/m²., more preferably the filtration flow may range from about 10 L/hour/m² to about 20 L/hour/m².

The Filtrate

The filtration process of the present invention results in a filtrate comprising VWF with high biological activity.

The VWF:Ag yield following filtration in the method of the invention is typically at least 50%, preferably at least 60%, or at least 70% or at least 75%.

The RCo VWF yield following filtration in the method of the invention is typically at least 40%, preferably at least 45%, at least 50% or at least 55%.

Preferably the ratio RCo VWF/Ag VWF in the filtrate obtained in step (b) of the method of the invention is at least 0.75, at least 0.8, at least 0.9, at least 1.0, at least 1.1, or at least 1.2.

In another embodiment, the ratio RCo VWF/Ag VWF in the filtrate obtained in step (b) is at least 75% of the ratio RCo VWF/Ag VWF in the solution provided in step (a). The ratio RCo VWF/Ag VWF decreases by less than 25% due to the filtration, preferably the decrease is less than 20%, or less than 15%, or less than 10% or less than 5%. More preferably, there is no decrease in the ratio RCo VWF/Ag VWF due to the filtration of the VWF solution. Most preferably, there is an increase in the ratio RCo VWF/Ag VWF due to the filtration.

In another embodiment, the filtrate obtained in step (b) comprises when analysed by multimer electrophoresis low multimers (1-5 bands), intermediate multimers (6-10 bands) and large multimers (HMWM, high molecular weight multimers, higher than 11 bands) of VWF. The terms “large multimers” and “high molecular weight multimers” are used synonymously herein if not indicated otherwise. Preferably, the relative amount of large multimers in the filtrate obtained in step (b) is at least 70%, at least 75%, at least 80%, or at least 85%, when compared to the total VWF content in the filtrate, respectively.

In yet another embodiment, the solution provided in step (a) as well as the filtrate obtained in step (b) comprises when analysed by multimer electrophoresis low multimers (1-5 bands), intermediate multimers (6-10 bands) and large multimers (HMWM, high molecular weight multimers, higher than 11 bands) of VWF. Preferably, the relative amount of large multimers in the filtrate obtained in step (b) is at least 70%, at least 75%, at least 80%, at least 85% when compared to the total VWF content in the solution provided in step (a) and in the filtrate obtained in step (b), respectively, according to this embodiment. In yet another preferred embodiment, said relative amount of large multimers in the filtrate obtained in step (b) is essentially identical to the relative amount of large multimers in the solution provided in step (a).

In another aspect, the present invention is a filtrated solution comprising VWF, obtainable by a process described herein. The filtrated solution typically has one or more of the properties described above, as regards VWF Ag activity, VWF RCo activity, and multimer content.

In another aspect, the present invention relates to a solution comprising VWF and arginine at a concentration of from 400 mM to 800 mM. Preferred arginine concentrations in the solution of the invention correspond to the preferred arginine concentrations in the solution to be filtrated as described above. Preferably, the solution further comprises calcium ions at a concentration of at least 100 mM. Preferred calcium ion concentrations in the solution of the invention correspond to the preferred calcium ion concentrations in the solution to be filtrated as described above. In other embodiments, the solution of the invention may comprise one or more further compounds which are optional components of the solution to be filtrated as described above.

Another aspect of the invention is a composition comprising VWF obtainable by a method described herein.

In yet another aspect the invention relates to a process of purifying VWF, comprising the method described hereinabove.

The VWF to be purified may be plasma-derived VWF or recombinantly produced VWF.

Recombinant VWF or variants thereof can be conveniently prepared using standard protocols. As one general example, recombinant VWF may be prepared by a procedure including one or more of the steps of: (a) preparing a construct comprising a polynucleotide sequence that encodes a protein and that is operably linked to at least one regulatory element; (b) introducing the construct into a host cell; (c) culturing the host cell to express the polypeptide and (d) collecting or isolating the polypeptide from the host cell. To express a VWF, a nucleotide sequence encoding the polypeptide, or a functional equivalent, may be inserted into an appropriate expression vector, i.e., a vector which contains the necessary elements for the transcription and translation of the inserted coding sequence. Methods which are well known to those skilled in the art may be used to construct expression vectors containing sequences encoding VWF and appropriate transcriptional and translational control elements. These methods include in vitro recombinant DNA techniques, synthetic techniques, and in vivo genetic recombination and are known in the art.

The VWF can be purified and characterized according to a variety of techniques known in the art. Exemplary systems for performing protein purification and analyzing protein purity include fast protein liquid chromatography (FPLC) (e.g., AKTA and Bio-Rad FPLC systems), hydrophobic interaction chromatography, and high-pressure liquid chromatography (HPLC). Exemplary chemistries for purification include ion exchange chromatography (e.g., Q, S), size exclusion chromatography, salt gradients, affinity purification (e.g., Ni, Co, FLAG, maltose, glutathione, protein NG), gel filtration, reverse-phase, ceramic HyperD® ion exchange chromatography, and hydrophobic interaction columns (HIC), among others known in the art. Also included are analytical methods such as SDS-PAGE (e.g., Coomassie, silver stain), preparative isoelectric focusing (IEF), immunoblot, Bradford, differential solubility (e.g., ammonium sulfate precipitation) and ELISA, which may be utilized during any step of the production or purification process, typically to measure the purity of the protein composition.

In certain aspects, the VWF can be subjected to multiple chromatographic purification steps, including any combination of affinity chromatography, ion-exchange chromatography, hydrophobic interaction chromatography, dye chromatography, hydroxyapatite chromatography, size exclusion chromatography and preferably immunoaffinity chromatography, mainly to concentrate the desired protein and to remove substances which may cause fragmentation, activation and/or degradation of the recombinant protein during manufacture, storage and/or use. Illustrative examples of such substances that are preferably removed by purification include other protein contaminants, such as modification enzymes like PACE/furin, VKOR, and VKGC; proteins, such as host cell proteins, which are released into the tissue culture media from the production cells during recombinant protein production; non-protein contaminants, such as lipids; and mixtures of protein and non-protein contaminants, such as lipoproteins. Purification procedures for VWF proteins are known in the art (see for example WO2011/022657A1).

In order to minimize the theoretical risk of virus contaminations, additional steps may be included in the process that allow effective inactivation or elimination of viruses. Such steps include, for example, heat treatment in the liquid or solid state, treatment with solvents and/or detergents, radiation in the visible or UV spectrum, gamma-radiation, and virus filtration.

The process for purifying VWF may comprise, in addition to the method of the invention, one or more of the following steps: cryoprecipitation, Al(OH)₃ adsorption, glycine precipitation, salt precipitation, pasteurization, dialysis, ultracentrifugation, sterile filtration, dilution, lyophilization and combinations thereof.

The VWF obtained by the methods and processes of the present invention can be formulated into pharmaceutical compositions. Suitable formulations are described in WO2015/188224A1 and WO2010/048275A2.

TABLE 3
Overview of the sequences in the sequence listing

| SEQ ID NO: | Description |
|---|---|
| 1 | cDNA sequence of human pre-pro-VWF |
| 2 | Amino acid sequence of human of pre-pro-VWF |

### EXAMPLES

### Example 1

Two different rVWF solutions designated “A” and “B”, respectively, were subjected to virus filtration. The rVWF was a VWF-albumin fusion, whereby the amino acid sequence has been described in WO2009/156137A1.

Solution “A” or “B” contained recombinantly expressed VWF and was obtained from an in house cell culture system and was purified separately.

Different amounts of arginine were added prior to filtration to give the final concentrations indicated in Table 4.

All VWF solutions had a pH of 6.8±0.1 when being subjected to virus filtration. All subsequent steps were performed at a room temperature of 23±5° C. The virus filtration was performed as a dead-end filtration. The starting intermediate (30-50 ml) for the virus filtration was filled in a pressure vessel and was then filtered through a 0.2/0.1 μm prefilter and a 20 nm filter (20N Planova; 0.001 m²) in series at a low input pressure of 0.3 bar (input pressure measured in front of prefilter). Pressure was obtained from compressed air. The 20N filtrate was collected in fractions followed by a postwash fraction. Aliquots of the filtrate fractions and of the postwash were pooled proportionally to the original fraction volume to represent the final sample of the filtration study (“preparation”) which was analyzed. The study is valid when the pre-use leakage test as well as the post-filtration integrity testing by post-use leakage test and gold particle test were passed.

TABLE 4

| Preparation | | Arg | CaCl₂ | Protein | Ag VWF | VWF:RCo | Protein | VWF:Ag | VWF:RCo |
|---|---|---|---|---|---|---|---|---|---|
| No. | Solution | [mM] | [mM] | [OD_{280-320]} | [IU/ml]* | [IU/ml]* | Yield [%] | Yield [%] | Yield [%] |
| 1 | B | 0 | 0 | 0.15 | 5.04 | 7.67 | 46.7 | 37.0 | 22.2 |
| 2 | B | 500 | 0 | 0.15 | 4.97 | 6.93 | 77.8 | 74.2 | 64.8 |
| 3 | A | 24 | 0 | 0.25 | 10.2 | 13.2 | 43.8 | 35.9 | 17.9 |
| 4 | A | 475 | 0 | 0.24 | 9.6 | 11.4 | 70.8 | 60.9 | 42.6 |

*prior to filtration

As can be seen from table 4, the presence of arginine in the rVWF solution leads to an increase in the yield of vWF:Ag and or VWF:RCo.

The filtrates from Preparation No. 1 and 2 were separated on a polyacrylamide gel and stained with Coomassie Blue. The gel was scanned and the bands were evaluated with ImageQuant software according to the manufacturer's instructions. The results are summarized in Table 5.

TABLE 5

| | Lane 1 | Lane 2 | Lane 3 | Lane 4 | Lane 5 | Lane 6 | Lane 7 | Lane 8 |
|---|---|---|---|---|---|---|---|---|
| Product | | rvWF | | | | | | |
| Preparation | / | 2 | | | 1 | | | / |
| Sample | SHP | pre | post | 20N | pre | post | 20N | SHP |
| | | 0.1 μm | 0.1 μm = | final | 0.1 μm | 0.1 μm = | final | |
| | | | pre 20N | | | pre 20N | | |
| Arg [mM] | / | 500 | | | 0 | | | / |
| Conc. [IU/mL] | 0.100 | 0.099 | 0.098 | 0.100 | 0.101 | 0.103 | 0.101 | 0.100 |
| % Multimer ≥11 | 102.18 | 105.71 | 102.89 | 102.03 | 99.74 | 85.16 | 74.75 | 97.82 |
| % Multimer 6-10 | 98.9 | 91.7 | 96.9 | 99.4 | 100.2 | 88.2 | 72.8 | 101.14 |
| % Multimer 1-5 | 99.4 | 102.1 | 100.4 | 99.2 | 100.0 | 116.9 | 133.5 | 100.57 |

As can be seen, the filtrate of the material with arginine contained a high proportion of high molecular weight multimers, whereas the sample without arginine had less HMWM.

### Example 2

There had been reports that calcium ions had a positive effect when subjecting VWF to virus filtration (VF). It was therefore investigated whether arginine could improve the VWF yield also in the presence of calcium ions.

The filtration conditions were as described in Example 1 except that a solution C was studied.

Solution “C” contained plasma-derived VWF and was obtained from a plasma protein manufacturing process.

Different amounts of CaCl₂) and arginine were added prior to filtration to give the final concentrations indicated in Table 6.

The filtrated volume was 36 ml in each Preparation.

The results are summarized in Table 6.

TABLE 6

| | | | | | | | Ratio RCo/ | Ratio RCo/ | Protein | FVIII IU | Ag vWF | VWF:RCo |
|---|---|---|---|---|---|---|---|---|---|---|---|---|
| | CaCl₂ | Arg | Ag vWF | VWF:RCo | Protein | dilution | Ag Prior | Ag after | yield | yield | yield | yield |
| Preparation | [mM] | [mM] | [IU/ml] | [IU/ml] | [OD] | factor | to VF | VF | [%] | [%] | [%] | [%] |
| 5 | 400 | 0 | 13.66 | 9.93 | 0.41 | 1:30 | 0.73 | 0.54 | 84.5 | 61.3 | 64.4 | 47.9 |
| 6 | 400 | 150 | 8.92 | n.a. | 0.24 | 1:50 | n.a. | n.a. | 95.3 | n.d. | 70.6 | n.a. |
| 7 | 400 | 300 | 12.01 | 8.82 | 0.41 | 1:30 | 0.73 | 0.59 | 83.8 | 71.2 | 72.0 | 58.1 |
| 8 | 400 | 300 | 6.6 | 6.28 | 0.25 | 1:50 | 0.95 | 0.94 | 91.1 | 61.9 | 83.8 | 84.5 |
| 9 | 400 | 400 | 8.24 | n.a. | 0.24 | 1:50 | n.a. | n.a. | 95.3 | n.d. | 83.5 | n.a. |
| 10 | 400 | 500 | 4.0 | 6.0 | 0.25 | 1:50 | 1.50 | 1.46 | 91.1 | 66.7 | 86.7 | 84.4 |
| 11 | 400 | 500 | 8.27 | n.a. | 0.24 | 1:50 | n.a. | n.a. | 95.3 | n.d. | 93.1 | n.a. |
| 12 | 400 | 750 | 8.89 | n.a. | 0.25 | 1:50 | n.a. | n.a. | 91.1 | n.d. | 80.7 | n.a. |
| 13* | 400 | 150 Lys | 9.18 | n.a. | 0.25 | 1:50 | n.a. | n.a. | 91.1 | n.d. | 65.1 | n.a. |
| 14** | 400 | 300 His | 10.64 | n.a. | 0.24 | 1:50 | n.a. | n.a. | 95.3 | n.d. | 83 | n.a. |

*lysine instead of arginine was added
**histidine instead of arginine was added

Preparation No. 5 in Table 6 shows that the VWF yield is higher in the presence of 400 mM CaCl₂) as compared to a solution without calcium ions (such as, e.g., Preparation No. 3 in Table 4 above). The further Preparations in Table 6, however, show that arginine further improved the VWF yield in virus filtration in the presence of calcium ions.

It was further shown that an improvement in the VWF yield can also be achieved by adding lysine or histidine instead of arginine.

1. A method of filtrating a solution comprising von Willebrand Factor (VWF), the method comprising the following steps:
(a) providing a solution comprising VWF and at least one basic amino acid, wherein the concentration of said at least one basic amino acid in the solution is at least 150 mM;
(b) subjecting the solution of step (a) to a virus filtration through a filter having a pore size of less than or equal to 35 nm.

2. The method of claim 1, wherein said VWF in the solution of step (a) comprises high molecular weight multimers (HMWM) of VWF.

3. The method according to claim 1, wherein the pressure during the virus filtration in step (b) is below 0.5 bar.

4. The method according to claim 1, wherein the pH of the solution provided in step (a) is between 5.0 and 9.0, in particular between 6.0 and 8.0.

5. The method according to claim 1, wherein the virus filtration in step (b) is conducted at a temperature between 15 and 30° C., in particular between 18 and 28° C.

6. The method according to claim 1, wherein the concentration of said at least one basic amino acid in the solution provided in step (a) is at least 300 mM, at least 350 mM, at least 400 mM, at least 450 mM or at least 500 mM.

7. The method according to claim 1, wherein the concentration of said at least one basic amino acid in the solution provided in step (a) is less than 1,000 mM, less than 950 mM, less than 900 mM, less than 850 mM, less than 800 mM or less than 750 mM.

8. The method according to claim 1, wherein the solution provided in step (a) further comprises calcium ions at a concentration of at least 50 mM, at least 100 mM, at least 200 mM, at least 300 mM or at least 350 mM.

9. The method according to claim 1, wherein the filter has a pore size of less than or equal to 25 nm or less than or equal to 20 nm.

10. The method according to claim 1, wherein the filter has a pore size of between 13 nm and 35 nm, between 13 nm and 25 nm, between 18 nm and 22 nm or between 13 nm and 17 nm.

11. The method according to claim 1, wherein the VWF is plasma-derived VWF or recombinantly obtained VWF.

12. The method according to claim 1, wherein the ratio RCo VWF/Ag VWF in the filtrate obtained in step (b) is at least 0.75, at least 0.8, at least 0.9, at least 1.0, at least 1.1, or at least 1.2.

13. The method according to claim 1, wherein the ratio RCo VWF/Ag VWF in the filtrate obtained in step (b) is at least 75% of the ratio RCo VWF/Ag VWF in the solution provided in step (a).

14. The method according to claim 1, wherein the VWF:Ag yield following filtration is at least 50%, at least 60%, at least 70% or at least 75%.

15. The method according to claim 1, wherein the RCo VWF yield following filtration is at least 40%, at least 45%, at least 50% or at least 55%.

16. The method according to claim 1, wherein the solution provided in step (a) as well as the filtrate obtained in step (b) comprises—when analysed by multimer electrophoresis—low multimers (1-5 bands), intermediate multimers (6-10 bands) and large multimers (HMWM, high molecular weight multimers, higher than 11 bands) of VWF, provided that the relative amount of large multimers in the filtrate obtained in step (b) is at least 70%, at least 75%, at least 80% or at least 85%, when compared to the total VWF content in the solution provided in step (a) and in the filtrate obtained in step (b), respectively.

17. The method according to claim 1, wherein said at least one amino acid is selected from the group consisting of arginine, lysine, histidine, ornithine and combinations thereof, preferably wherein the basic amino acid is arginine.

18. The method according to claim 1, wherein the solution provided in step (a) comprises Factor VIII (FVIII) in addition to VWF.

19. A filtrated solution of VWF obtainable by a method according to claim 1.

20. A composition comprising VWF obtainable by a method according to claim 1.

21. A process of purifying VWF, comprising the method of claim 1.


SEQUENCE LISTING (2 sequences)

SEQ ID NO: 1 - DNA, 8442 bp, Homo sapiens
  CDS (1)..(8442)
atgattcctgccagatttgccggggtgctgcttgctctggccctcattttgccagggaccctttgtgcagaaggaactcgcggcaggtcatccacggcccgatgcagccttttcggaagtgacttcgtcaacacctttgatgggagcatgtacagctttgcgggatactgcagttacctcctggcagggggctgccagaaacgctccttctcgattattggggacttccagaatggcaagagagtgagcctctccgtgtatcttggggaattttttgacatccatttgtttgtcaatggtaccgtgacacagggggaccaaagagtctccatgccctatgcctccaaagggctgtatctagaaactgaggctgggtactacaagctgtccggtgaggcctatggctttgtggccaggatcgatggcagcggcaactttcaagtcctgctgtcagacagatacttcaacaagacctgcgggctgtgtggcaactttaacatctttgctgaagatgactttatgacccaagaagggaccttgacctcggacccttatgactttgccaactcatgggctctgagcagtggagaacagtggtgtgaacgggcatctcctcccagcagctcatgcaacatctcctctggggaaatgcagaagggcctgtgggagcagtgccagcttctgaagagcacctcggtgtttgcccgctgccaccctctggtggaccccgagccttttgtggccctgtgtgagaagactttgtgtgagtgtgctggggggctggagtgcgcctgccctgccctcctggagtacgcccggacctgtgcccaggagggaatggtgctgtacggctggaccgaccacagcgcgtgcagcccagtgtgccctgctggtatggagtataggcagtgtgtgtccccttgcgccaggacctgccagagcctgcacatcaatgaaatgtgtcaggagcgatgcgtggatggctgcagctgccctgagggacagctcctggatgaaggcctctgcgtggagagcaccgagtgtccctgcgtgcattccggaaagcgctaccctcccggcacctccctctctcgagactgcaacacctgcatttgccgaaacagccagtggatctgcagcaatgaagaatgtccaggggagtgccttgtcacaggtcaatcacacttcaagagctttgacaacagatacttcaccttcagtgggatctgccagtacctgctggcccgggattgccaggaccactccttctccattgtcattgagactgtccagtgtgctgatgaccgcgacgctgtgtgcacccgctccgtcaccgtccggctgcctggcctgcacaacagccttgtgaaactgaagcatggggcaggagttgccatggatggccaggacgtccagctccccctcctgaaaggtgacctccgcatccagcatacagtgacggcctccgtgcgcctcagctacggggaggacctgcagatggactgggatggccgcgggaggctgctggtgaagctgtcccccgtctatgccgggaagacctgcggcctgtgtgggaattacaatggcaaccagggcgacgacttccttaccccctctgggctggcggagccccgggtggaggacttcgggaacgcctggaagctgcacggggactgccaggacctgcagaagcagcacagcgatccctgcgccctcaacccgcgcatgaccaggttctccgaggaggcgtgcgcggtcctgacgtcccccacattcgaggcctgccatcgtgccgtcagcccgctgccctacctgcggaactgccgctacgacgtgtgctcctgctcggacggccgcgagtgcctgtgcggcgccctggccagctatgccgcggcctgcgcggggagaggcgtgcgcgtcgcgtggcgcgagccaggccgctgtgagctgaactgcccgaaaggccaggtgtacctgcagtgcgggaccccctgcaacctgacctgccgctctctctcttacccggatgaggaatgcaatgaggcctgcctggagggctgcttctgccccccagggctctacatggatgagaggggggactgcgtgcccaaggcccagtgcccctgttactatgacggtgagatcttccagccagaagacatcttctcagaccatcacaccatgtgctactgtgaggatggcttcatgcactgtaccatgagtggagtccccggaagcttgctgcctgacgctgtcctcagcagtcccctgtctcatcgcagcaaaaggagcctatcctgtcggccccccatggtcaagctggtgtgtcccgctgacaacctgcgggctgaagggctcgagtgtaccaaaacgtgccagaactatgacctggagtgcatgagcatgggctgtgtctctggctgcctctgccccccgggcatggtccggcatgagaacagatgtgtggccctggaaaggtgtccctgcttccatcagggcaaggagtatgcccctggagaaacagtgaagattggctgcaacacttgtgtctgtcgggaccggaagtggaactgcacagaccatgtgtgtgatgccacgtgctccacgatcggcatggcccactacctcaccttcgacgggctcaaatacctgttccccggggagtgccagtacgttctggtgcaggattactgcggcagtaaccctgggacctttcggatcctagtggggaataagggatgcagccacccctcagtgaaatgcaagaaacgggtcaccatcctggtggagggaggagagattgagctgtttgacggggaggtgaatgtgaagaggcccatgaaggatgagactcactttgaggtggtggagtctggccggtacatcattctgctgctgggcaaagccctctccgtggtctgggaccgccacctgagcatctccgtggtcctgaagcagacataccaggagaaagtgtgtggcctgtgtgggaattttgatggcatccagaacaatgacctcaccagcagcaacctccaagtggaggaagaccctgtggactttgggaactcctggaaagtgagctcgcagtgtgctgacaccagaaaagtgcctctggactcatcccctgccacctgccataacaacatcatgaagcagacgatggtggattcctcctgtagaatccttaccagtgacgtcttccaggactgcaacaagctggtggaccccgagccatatctggatgtctgcatttacgacacctgctcctgtgagtccattggggactgcgcctgcttctgcgacaccattgctgcctatgcccacgtgtgtgcccagcatggcaaggtggtgacctggaggacggccacattgtgcccccagagctgcgaggagaggaatctccgggagaacgggtatgagtgtgagtggcgctataacagctgtgcacctgcctgtcaagtcacgtgtcagcaccctgagccactggcctgccctgtgcagtgtgtggagggctgccatgcccactgccctccagggaaaatcctggatgagcttttgcagacctgcgttgaccctgaagactgtccagtgtgtgaggtggctggccggcgttttgcctcaggaaagaaagtcaccttgaatcccagtgaccctgagcactgccagatttgccactgtgatgttgtcaacctcacctgtgaagcctgccaggagccgggaggcctggtggtgcctcccacagatgccccggtgagccccaccactctgtatgtggaggacatctcggaaccgccgttgcacgatttctactgcagcaggctactggacctggtcttcctgctggatggctcctccaggctgtccgaggctgagtttgaagtgctgaaggcctttgtggtggacatgatggagcggctgcgcatctcccagaagtgggtccgcgtggccgtggtggagtaccacgacggctcccacgcctacatcgggctcaaggaccggaagcgaccgtcagagctgcggcgcattgccagccaggtgaagtatgcgggcagccaggtggcctccaccagcgaggtcttgaaatacacactgttccaaatcttcagcaagatcgaccgccctgaagcctcccgcatcgccctgctcctgatggccagccaggagccccaacggatgtcccggaactttgtccgctacgtccagggcctgaagaagaagaaggtcattgtgatcccggtgggcattgggccccatgccaacctcaagcagatccgcctcatcgagaagcaggcccctgagaacaaggccttcgtgctgagcagtgtggatgagctggagcagcaaagggacgagatcgttagctacctctgtgaccttgcccctgaagcccctcctcctactctgcccccccacatggcacaagtcactgtgggcccggggctcttgggggtttcgaccctggggcccaagaggaactccatggttctggatgtggcgttcgtcctggaaggatcggacaaaattggtgaagccgacttcaacaggagcaaggagttcatggaggaggtgattcagcggatggatgtgggccaggacagcatccacgtcacggtgctgcagtactcctacatggtgaccgtggagtaccccttcagcgaggcacagtccaaaggggacatcctgcagcgggtgcgagagatccgctaccagggcggcaacaggaccaacactgggctggccctgcggtacctctctgaccacagcttcttggtcagccagggtgaccgggagcaggcgcccaacctggtctacatggtcaccggaaatcctgcctctgatgagatcaagaggctgcctggagacatccaggtggtgcccattggagtgggccctaatgccaacgtgcaggagctggagaggattggctggcccaatgcccctatcctcatccaggactttgagacgctcccccgagaggctcctgacctggtgctgcagaggtgctgctccggagaggggctgcagatccccaccctctcccctgcacctgactgcagccagcccctggacgtgatccttctcctggatggctcctccagtttcccagcttcttattttgatgaaatgaagagtttcgccaaggctttcatttcaaaagccaatatagggcctcgtctcactcaggtgtcagtgctgcagtatggaagcatcaccaccattgacgtgccatggaacgtggtcccggagaaagcccatttgctgagccttgtggacgtcatgcagcgggagggaggccccagccaaatcggggatgccttgggctttgctgtgcgatacttgacttcagaaatgcatggggcgcgcccgggagcctcaaaggcggtggtcatcctggtcacggacgtctctgtggattcagtggatgcagcagctgatgccgccaggtccaacagagtgacagtgttccctattggaattggagatcgctacgatgcagcccagctacggatcttggcaggcccagcaggcgactccaacgtggtgaagctccagcgaatcgaagacctccctaccatggtcaccttgggcaattccttcctccacaaactgtgctctggatttgttaggatttgcatggatgaggatgggaatgagaagaggcccggggacgtctggaccttgccagaccagtgccacaccgtgacttgccagccagatggccagaccttgctgaagagtcatcgggtcaactgtgaccgggggctgaggccttcgtgccctaacagccagtcccctgttaaagtggaagagacctgtggctgccgctggacctgcccctgcgtgtgcacaggcagctccactcggcacatcgtgacctttgatgggcagaatttcaagctgactggcagctgttcttatgtcctatttcaaaacaaggagcaggacctggaggtgattctccataatggtgcctgcagccctggagcaaggcagggctgcatgaaatccatcgaggtgaagcacagtgccctctccgtcgagctgcacagtgacatggaggtgacggtgaatgggagactggtctctgttccttacgtgggtgggaacatggaagtcaacgtttatggtgccatcatgcatgaggtcagattcaatcaccttggtcacatcttcacattcactccacaaaacaatgagttccaactgcagctcagccccaagacttttgcttcaaagacgtatggtctgtgtgggatctgtgatgagaacggagccaatgacttcatgctgagggatggcacagtcaccacagactggaaaacacttgttcaggaatggactgtgcagcggccagggcagacgtgccagcccatcctggaggagcagtgtcttgtccccgacagctcccactgccaggtcctcctcttaccactgtttgctgaatgccacaaggtcctggctccagccacattctatgccatctgccagcaggacagttgccaccaggagcaagtgtgtgaggtgatcgcctcttatgcccacctctgtcggaccaacggggtctgcgttgactggaggacacctgatttctgtgctatgtcatgcccaccatctctggtttataaccactgtgagcatggctgtccccggcactgtgatggcaacgtgagctcctgtggggaccatccctccgaaggctgtttctgccctccagataaagtcatgttggaaggcagctgtgtccctgaagaggcctgcactcagtgcattggtgaggatggagtccagcaccagttcctggaagcctgggtcccggaccaccagccctgtcagatctgcacatgcctcagcgggcggaaggtcaactgcacaacgcagccctgccccacggccaaagctcccacgtgtggcctgtgtgaagtagcccgcctccgccagaatgcagaccagtgctgccccgagtatgagtgtgtgtgtgacccagtgagctgtgacctgcccccagtgcctcactgtgaacgtggcctccagcccacactgaccaaccctggcgagtgcagacccaacttcacctgcgcctgcaggaaggaggagtgcaaaagagtgtccccaccctcctgccccccgcaccgtttgcccacccttcggaagacccagtgctgtgatgagtatgagtgtgcctgcaactgtgtcaactccacagtgagctgtccccttgggtacttggcctcaaccgccaccaatgactgtggctgtaccacaaccacctgccttcccgacaaggtgtgtgtccaccgaagcaccatctaccctgtgggccagttctgggaggagggctgcgatgtgtgcacctgcaccgacatggaggatgccgtgatgggcctccgcgtggcccagtgctcccagaagccctgtgaggacagctgtcggtcgggcttcacttacgttctgcatgaaggcgagtgctgtggaaggtgcctgccatctgcctgtgaggtggtgactggctcaccgcggggggactcccagtcttcctggaagagtgtcggctcccagtgggcctccccggagaacccctgcctcatcaatgagtgtgtccgagtgaaggaggaggtctttatacaacaaaggaacgtctcctgcccccagctggaggtccctgtctgcccctcgggctttcagctgagctgtaagacctcagcgtgctgcccaagctgtcgctgtgagcgcatggaggcctgcatgctcaatggcactgtcattgggcccgggaagactgtgatgatcgatgtgtgcacgacctgccgctgcatggtgcaggtgggggtcatctctggattcaagctggagtgcaggaagaccacctgcaacccctgccccctgggttacaaggaagaaaataacacaggtgaatgttgtgggagatgtttgcctacggcttgcaccattcagctaagaggaggacagatcatgacactgaagcgtgatgagacgctccaggatggctgtgatactcacttctgcaaggtcaatgagagaggagagtacttctgggagaagagggtcacaggctgcccaccctttgatgaacacaagtgtctggctgagggaggtaaaattatgaaaattccaggcacctgctgtgacacatgtgaggagcctgagtgcaacgacatcactgccaggctgcagtatgtcaaggtgggaagctgtaagtctgaagtagaggtggatatccactactgccagggcaaatgtgccagcaaagccatgtactccattgacatcaacgatgtgcaggaccagtgctcctgctgctctccgacacggacggagcccatgcaggtggccctgcactgcaccaatggctctgttgtgtaccatgaggttctcaatgccatggagtgcaaatgctcccccaggaagtgcagcaagtga

SEQ ID NO: 2 - protein, 2813 aa, Homo sapiens
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDVQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRIALLLMASQEPQRMSRNFVRYVQGLKKKKVIVIPVGIGPHANLKQIRLIEKQAPENKAFVLSSVDELEQQRDEIVSYLCDLAPEAPPPTLPPHMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQPDGQTLLKSHRVNCDRGLRPSCPNSQSPVKVEETCGCRWTCPCVCTGSSTRHIVTFDGQNFKLTGSCSYVLFQNKEQDLEVILHNGACSPGARQGCMKSIEVKHSALSVELHSDMEVTVNGRLVSVPYVGGNMEVNVYGAIMHEVRFNHLGHIFTFTPQNNEFQLQLSPKTFASKTYGLCGICDENGANDFMLRDGTVTTDWKTLVQEWTVQRPGQTCQPILEEQCLVPDSSHCQVLLLPLFAECHKVLAPATFYAICQQDSCHQEQVCEVIASYAHLCRTNGVCVDWRTPDFCAMSCPPSLVYNHCEHGCPRHCDGNVSSCGDHPSEGCFCPPDKVMLEGSCVPEEACTQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWASPENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEPECNDITARLQYVKVGSCKSEVEVDIHYCQGKCASKAMYSIDINDVQDQCSCCSPTRTEPMQVALHCTNGSVVYHEVLNAMECKCSPRKCSK